(19) United States
(12) Patent Application Publication
(10) Pub. No.: US 2018/0104685 A1
(43) Pub. Date: Apr. 19, 2018

(54) MICROFLUIDIC DEVICES AND METHODS FOR PATHOGEN DETECTION IN LIQUID SAMPLES

(71) Applicant: The Administrators of the Tulane Educational Fund, New Orleans, LA (US)

(72) Inventors: Jason RYANS, New Orleans, LA (US); Ashwin SIVAKUMAR, New Orleans, LA (US); Christopher DAVITT, New Orleans, LA (US)

(21) Appl. No.: 15/797,102

(22) Filed: Oct. 30, 2017

Related U.S. Application Data
(63) Continuation-in-part of application No. PCT/US2016/030174, filed on Apr. 29, 2016.
(60) Provisional application No. 62/154,535, filed on Apr. 29, 2015.

(51) Int. Cl. B01L 3/00; G01N 33/558; G01N 33/569; G01N 33/58

(52) U.S. Cl. CPC B01L 3/5027; G01N 33/558; G01N 33/569; G01N 33/587; G01N 2333/185; B01L 2300/0636; B01L 2300/0864; B01L 2300/126; B01L 2200/10

(57) ABSTRACT

One aspect of the present disclosure relates to a device for detecting a pathogen biomarker in a biological sample. The device can comprise a substrate. The substrate can comprise paper. The device can also comprise a hydrophobic material applied to the substrate to define at least one target region, a sample region, and at least one channel in fluid communication with the at least one target region and the sample region. The device can further include a predetermined amount of antibodies that specifically bind to at least one pathogen biomarker provided in the at least one target region. The predetermined amount of antibodies is conjugated with colloidal gold.

### CROSS-REFERENCE TO RELATED APPLICATIONS

This application claims priority to International Patent Application No. PCT/US2016/030174, filed on Apr. 29, 2016, which claims priority to U.S. Provisional Application Ser. No. 62/154,535, filed Apr. 29, 2015, both of which are hereby incorporated by reference in its entirety.

### TECHNICAL FIELD

The present disclosure relates generally to devices and methods for detecting a pathogen biomarker in a liquid sample and, more particularly, to highly sensitive and specific microfluidic devices and methods for rapidly detecting a target pathogen biomarker in a liquid sample.

### BACKGROUND

To diagnose disease, biological samples (e.g., blood, urine, etc.) are tested for pathogens, with the ultimate goal of determining the most effective treatment for a particular disease or disease state and monitoring the progression of the disease in the patient. Low quantities of pathogen in biological samples make their detection difficult to perform rapidly, cheaply and accurately. Detection is additionally confounded by other non-pathogens in the sample (e.g., red blood cells, dust) that can reduce the signal-to-background ratio during pathogen detection. Traditional methods for overcoming these drawbacks are growth-based (e.g., multiplying the pathogen for 12-16 hours so they can be easily detected followed by further testing of pathogen characteristics) or DNA-based. Such growth-based methods are slow and expensive, while the DNA-based tests, which are more rapid than the growth-based methods, are comparatively more expensive and, being genotypic, may not predict phenotypic relevance.

### SUMMARY

The present disclosure relates generally to devices and methods for detecting a pathogen biomarker in a liquid sample and, more particularly, to highly sensitive and specific microfluidic devices and methods for rapidly detecting a pathogen biomarker in a liquid sample.

One aspect of the present disclosure relates to a microfluidic device for detecting a pathogen biomarker in a liquid sample. The device can comprise a substrate. The device can also comprise a hydrophobic material applied to the substrate to define at least one target region, a sample region, and at least one channel in fluid communication with the at least one target region and the sample region. The device can further include a predetermined amount of antibodies that specifically bind to at least one pathogen biomarker provided in the at least one target region. The predetermined amount of antibodies is conjugated with colloidal gold.

Another aspect of the present disclosure relates to a method for detecting the presence of at least one pathogen in a subject. One step of the method can include obtaining a biological sample from the subject. The biological sample can then be applied to a microfluidic device. The microfluidic device can comprise a substrate. The device can also comprise a hydrophobic material applied to the substrate to define at least one target region, a sample region, and at least one channel in fluid communication with the at least one target region and the sample region. The device can further include a predetermined amount of antibodies that specifically bind to at least one pathogen biomarker provided in the at least one target region. The predetermined amount of antibodies is conjugated with colloidal gold. Next, the presence of the at least one pathogen biomarker in the biological sample can be determined. The at least one pathogen biomarker, if present in the biological sample, specifically binds to the predetermined amount of antibodies conjugated with colloidal gold in the target region causing the formation of a colloidal gold conglomerate. The presence of a colloidal gold conglomerate is indicative of the presence of the at least one pathogen in the subject during the determining step.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic illustration showing an upper surface of a microfluidic device for detecting at least one pathogen biomarker in a biological sample constructed in accordance with one aspect of the present disclosure;

FIG. 2 is a schematic illustration showing a side view of the microfluidic device of FIG. 1;

FIG. 3 is a top view of a multichannel microfluidic device for detecting at least one pathogen biomarker in a biological sample constructed in accordance with another aspect of the present disclosure;

FIG. 4 is a schematic illustration showing an upper surface of a microfluidic device for detecting at least one pathogen biomarker in a biological sample constructed in accordance with one aspect of the present disclosure;

FIG. 5 is a process flow diagram illustrating a method for detecting a pathogen biomarker in a biological sample according to another aspect of the present disclosure;

FIG. 6 is a process flow diagram illustrating a method for detecting a pathogen biomarker in a biological sample according to another aspect of the present disclosure;

FIG. 7 is an image showing that un-reacted colloidal gold nanoparticles (AuNPs) (left) appear the same as anti-Human IgG conjugated AuNPs (right) following conjugation in 0.01×PBS. This suggests that 0.01×PBS is an appropriate medium for conjugation of antibodies to AuNPs while maintaining the appearance of the particles in suspension.

FIG. 8 is an image showing that the structure of AuNPs are not altered following conjugation to anti-Human IgG (right) compared to un-conjugated AuNPs (left). Darkened areas around the anti-human IgG-conjugated AuNPs suggests successful attachment of the Ab to the NPs. Therefore the use of 0.01×PBS is a suitable medium for the attachment of biotin-conjugated antibodies to streptavidin-AuNPs.

FIG. 9 is an image showing that AuNPs can recognize and initiate a color change upon the binding to human IgG in an in vitro test.

FIGS. 10A and 10B provide a top (A) and side (B) view of an immunoassay including a sample pad and a positive control, wherein the immunoassay is exhibiting a positive result.

FIGS. 11A and 11B provide a top (A) and side (B) view of an immunoassay including a sample pad and a positive control, wherein the immunoassay is exhibiting a negative result.

FIGS. 12A-C provide a top view A and a bottom view B, as well as a side view C, of an immunoassay including both a positive and negative control and perpendicular channels.

### DETAILED DESCRIPTION

In the following detailed description, reference is made to the accompanying drawings that form a part hereof, and in which are shown by way of illustration specific embodiments in which the invention may be practiced. It is understood that other embodiments may be utilized and structural changes may be made without departing from the scope of the present invention.

### Definitions

Unless defined otherwise, all technical and scientific terms used herein have the same meaning as is commonly understood by one of skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the apparatus in use or operation in addition to the orientation depicted in the figures. For example, if the apparatus in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

As used herein, the terms “about” or “approximately” can generally mean within 20 percent, preferably within 10 percent, and more preferably within 5 percent of a given value or range. Numerical quantities given herein are approximate, meaning that the term “about” or “approximately” can be inferred if not expressly stated.

As used herein, the term “pathogen biomarker” can refer to a substance in a biological sample indicative of a pathogen capable of being detected and analyzed by the present disclosure. Pathogen biomarkers can include, but are not limited to, molecules, peptides, proteins (including prions), nucleic acids, oligonucleotides, cells, pathogens (e.g., viruses, bacteria, fungi), fragments of pathogens, products or biomolecules associated with and/or indicative of pathogens (e.g., enzymes or metabolic products produced by pathogen), and any substance (e.g., antigens) indicative of a pathogen for which attachment sites, binding members, or receptors can be developed. A pathogen biomarker can also refer to a protein, such as an antibody, produced by a mammalian subject in response to the presence or activity of at least one pathogen in the subject and that specifically binds to a pathogen antigen.

As used herein, the terms “specific binding” or “specifically binding”, refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally

The term “antigen” as used herein refer to a portion or portions of molecules which are capable of inducing a specific immune response in a subject alone or in combination with an adjuvant. The term “epitope,” as used herein, refers to a portion of a polypeptide having antigenic or immunogenic activity in an animal, for example a mammal, for example, a human.

The term “antibody” as used herein refers to immunoglobulin molecules or other molecules which comprise at least one antigen-binding domain. The term “antibody” as used herein is intended to include whole antibodies, monoclonal antibodies, polyclonal antibodies, chimeric antibodies, humanized antibodies, primatized antibodies, multi-specific antibodies, single chain antibodies, epitope-binding fragments, e.g., Fab, Fab′ and F(ab′)2, Fd, Fvs, single-chain Fvs (scFv), disulfide-linked Fvs (sdFv), fragments comprising either a VL or VH domain, and totally synthetic and recombinant antibodies. The antibodies can be of any type (e.g., IgG, IgE, IgM, IgD, IgA, and IgY), class (e.g., IgG1, IgG2, IgG3, IgG4, IgA1 and IgA2) or subclass of immunoglobulin molecule.

The term “antibody fragment” or “binding fragment” as used herein is intended to include any appropriate antibody fragment which comprises an antigen-binding domain that displays antigen binding function. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, T and Abs, ds-scFv, dimers, minibodies, diabodies, bispecific antibody fragments and other fragments can also be synthesized by recombinant techniques or can be chemically synthesized. Techniques for producing antibody fragments are well known and described in the art. Antibody fragments, including single-chain antibodies, may comprise the variable region(s) alone or in combination with the entirety or a portion of the following: hinge region, CH1, CH2, and CH3 domains.

In the context of various embodiments, the term “nanoparticle” may refer to an object of a size less than about 1 micron or 1 μm. In one example, a gold nanoparticle may be about 10 nm to about 1000 nm in size, in various embodiments, the gold nanoparticle may be about 40 nm in size. Depending on the shape of the nanoparticle, the size relates to the diameter or length of the respective structure. In various embodiments, the size is the mean particle size. A gold nanoparticle may be selected from the group consisting of a gold nanosphere, a gold nanorod, a gold nanotube, a gold nanosheil, a gold nanodot and a gold nanowire.

As used herein, the term “colloidal gold nanoparticles” refers to gold nanoparticles capable of forming a colloid. A colloid may be analogous to a solution: both are systems of molecules, atoms or particles in a solvent. The nanoparticles of a colloidal system, however, because of their size (typically in nanometers) or the distance between them (also typically in nanometers), and their solid cores, may attract one another with sufficient force to make them tend to aggregate even when the only means of transport for the nanoparticles in the solvent is diffusion. A “colloidal gold nanoparticle” may not be itself a colloid but rather only a constituent of a colloid. Nonetheless, the term “colloid” may be used to denote the nanoparticle itself.

As used herein, the term “biological sample” can refer to any quantity of a tissue, liquid or fluid obtained and/or derived from a subject that contains, or is suspected of containing, one or more pathogen biomarkers. In some instances, a biological sample can comprise a bodily fluid, such as serum, serum, buffy coat, saliva, whole blood, partially processed blood, nasopharyngeal fluid (e.g., sinus drainage), wound exudates, pus, lung and other respiratory aspirates, bronchial lavage fluids, medial and inner ear aspirates, cyst aspirates, cerebrospinal fluid, stool, diarrheal fluid, tears, mammary secretions, ovarian contents, ascites fluid, mucous, gastric fluid, gastrointestinal contents, urethral discharge, peritoneal fluid, meconium, vaginal fluid or discharge, amniotic fluid, semen, penile discharge, synovial fluid, urine, sputum, seminal or lymph fluids, or the like. In some embodiments, the biological sample is a mucous-containing sample, such as saliva or sputum. A biological sample, such as a liquid sample can be first processed (e.g., purified or partially purified) and/or mixed with buffers and/or reagents used to generate appropriate assay conditions.

A biological sample may be fresh or stored (e.g. blood or blood fraction stored in a blood bank). Samples can be stored for varying amounts of time, such as being stored for an hour, a day, a week, a month, or more than a month. The biological sample may be a bodily fluid expressly obtained for the assays of this invention or a bodily fluid obtained for another purpose which can be sub-sampled in order to carry out the method.

The terms “individual,” “subject,” and “patient” can be used interchangeably herein irrespective of whether the subject has or is currently undergoing any form of treatment. As used herein, the term “subject” generally refers to any vertebrate, including, but not limited to a mammal. Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). Treatment of humans is of particular interest.

As used herein, the term “in fluid communication” can refer to a fluid (e.g., a liquid) that can move from one part of a device to another part of the device. Two or more parts of the device can be in fluid communication by being physically linked together or adjacent one another, or the fluid communication can be mediated through another part of the device.

As used herein, the term “coupled” can refer to direct coupling or indirect coupling via a separate object. The term can also encompass two or more components that are continuous with one another by virtue of each of the components being formed from the same piece of material. Also, the term “coupled” may include chemical, mechanical, thermal or electrical coupling. Fluid coupling can mean that fluid is in communication between designated parts or locations.

As used herein, the term “point-of-care environment” can refer to real-time diagnostic testing that can be done in a rapid time frame so that the resulting test is performed faster than comparable tests that do not employ the present disclosure. Point-of-care environments can include, but are not limited to: emergency rooms; at a bedside; in a stat laboratory; operating rooms; hospital laboratories and other clinical laboratories; doctor's offices; in the field; or in any situation or locale where a rapid and accurate result is desired.

### Overview

The present disclosure relates generally to devices and methods for detecting a pathogen biomarker in a biological sample and, more particularly, to highly sensitive and specific microfluidic devices and methods for rapidly detecting a pathogen biomarker in a biological sample. Conventional techniques and associated diagnostic devices for detecting pathogens (e.g., viruses and bacteria) in blood require a full microbiological laboratory and can entail a lengthy process of partially classifying the particular pathogen followed by subsequent culture before accurate detection can be done. Unlike conventional techniques, the present disclosure directly detects pathogens, or biomolecules associated therewith, and either reduces or obviates the need to incubate or culture the pathogens prior to detection. Advantageously, the present disclosure provides rapid, point-of-care pathogen detection with exceptional sensitivity and specificity while utilizing small amounts of biological samples and sample preparation, thereby allowing a clinician or other medical professional to quickly guide treatment. The present disclosure also allows for the detection of a wide range of pathogens with a visible colorimetric readout that does not require optically transparent biological samples. In addition, the present disclosure advantageously allows for the simultaneous detection of various distinct pathogens (e.g., various genetically distinct viruses from the same species) using the same device, thereby further facilitating quick and appropriate treatment. These and other advantages of the present disclosure are discussed in more detail below.

### Devices

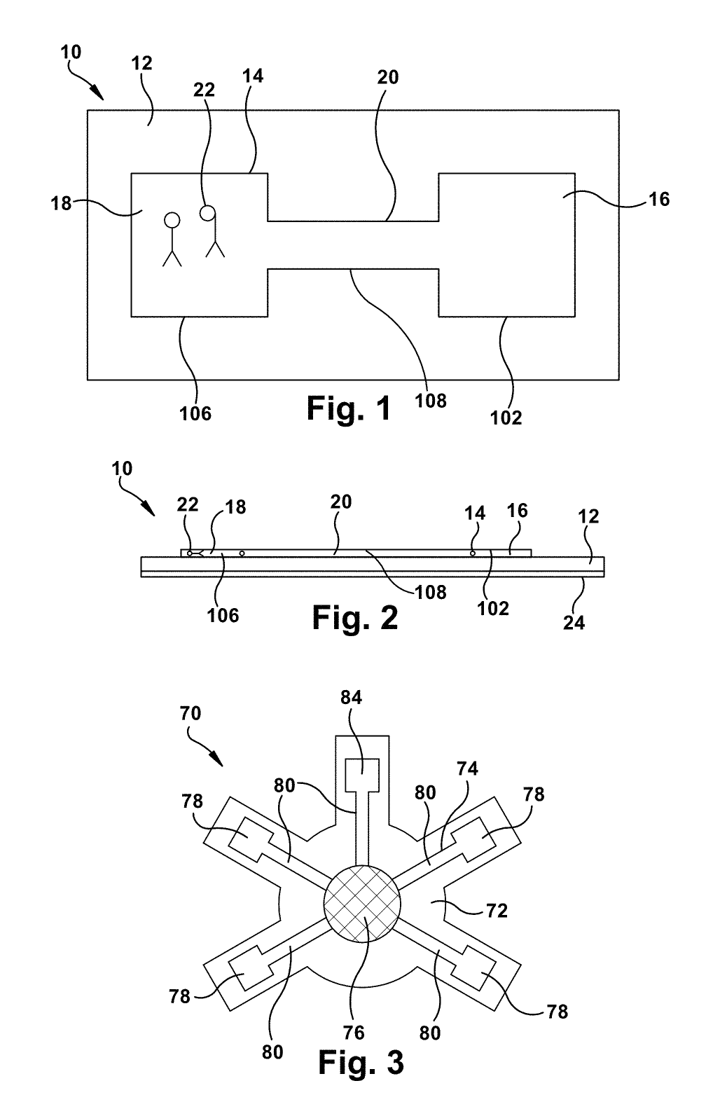

One aspect of the present disclosure can include a microfluidic device 10 (FIGS. 1-2) for detecting at least one pathogen biomarker in a biological sample. The microfluidic device 10 can generally comprise a substrate 12, a hydrophobic material 14 applied to the substrate 12, and a predetermined amount of antibodies 22 that specifically bind to at least one pathogen biomarker in the biological sample. In some instances, the predetermined amount of antibodies 22 can be conjugated with colloidal gold nanoparticles. In other instances, the predetermined amount of antibodies 22 can be conjugated with silver nanoparticles, a peroxidase enzyme, fluorescent molecules and/or luminescent molecules. The hydrophobic material 14 applied to the substrate can 12 define a sample region 16 for receiving a biological sample, at least one target region 18 for providing the predetermined amount of antibodies 22, and at least one channel 20 that is in fluid communication with both the sample region 16 and the target region 18.

By “microfluidic” it is meant that the device 10 can include one or more sets of channels 20 that interconnect to form a generally closed microfluidic network. Generally, microfluidic channels can include fluid passages having at least one internal cross-sectional dimension that is less than about 500 μm (e.g., typically between about 0.1 μm and about 500 μm) and/or a height or width of less than about 200, 100 or 50 μm. Such a microfluidic network may include one, two, or more openings, (e.g., a sample region 16 and the target region 18) at network termini, or intermediate to the network that interface with the external environment. Such openings may receive, store, and/or dispense a liquid. A microfluidic device may also include any other suitable features or mechanisms that contribute to liquid, reagent, and/or target analyte (e.g., a pathogen biomarker) manipulation or analysis. Furthermore, a microfluidic device can include one or more features (e.g., any detectable shape or symbol, or set of shapes or symbols, such as black-and-white or colored barcode, a word, a number, and/or the like, that has a distinctive position, identity, and/or other property) that act as a code to identify a particular target pathogen biomarker or a control.

Although some of the embodiments of the device 10 are described below as having a sample region 16, a single target region 18, and a single channel 20 (a “single channel device”), it will be appreciated that the device can have any number, combination, and arrangement of sample regions 16, target regions 18, and channels 20 (a “multi-channel device”), for example, to facilitate the simultaneous detection of multiple pathogenic biomarkers using a single device 10.

In some instances, the device 10 can be configured as a single, standalone platform for detecting a pathogen biomarker that is free from physical connection to any other apparatus or devices. In other instances, multiple devices 10 can be formed or located on a substrate (e.g., a plastic sheet) such that the substrate defines a plurality of sections, each of which includes a device of the present disclosure. In such instances, each section can be selectively removed (e.g., broken off) from the substrate as needed for analysis. Alternatively, the substrate could be processed using an automated machine for multiplex analysis.

As shown in FIGS. 1-2, the substrate 12 can have a rectangular shape. Although the substrate 12 is depicted in FIGS. 1-2 as having a rectangular shape, it will be appreciated that the substrate 12 can have any desired shape (e.g., rectangular, puck-shaped, gear-shaped, star-shaped etc.). The dimensions (e.g., height, width, length) of the substrate 12 can be varied as needed.

The substrate 12 of the microfluidic device 10 can be fabricated from an efficient liquid-transferring material that allows a biological sample to be placed on the sample region 16 and freely flow to the target region 18. In one example, a substrate 12 can be fabricated from a paper material or sintered polymer. A substrate 12 fabricated from a paper material offers the advantage of being inexpensive, lightweight, available in a wide range of thickness, and is disposable. Thus, paper-based microfluidic devices are suitable for the development of diagnostic assays in developing countries and harsh environments. Aqueous biological sample solutions can be transported by wicking (i.e., capillary action), thus realizing passive pumping. In addition, well-defined pore sizes in paper can be manufactured and suspended solids (e.g., clotted red blood cells) within biological samples can be separated based on size exclusion before an assay is performed. Paper is biocompatible with various biological samples and can thus be modified with a wide range of functional groups to enable covalent bonding of proteins, DNA, or small molecules. In some instances, the device 10 can include a backing layer 24 to provide support for the substrate 12. The backing layer 24 may be Mylar or other rigid support material.

In order to manipulate fluids along the desired direction in the substrate 12 (e.g., paper), hydrophobic barrier materials can be applied (e.g., patterned) to the substrate 12, thereby defining the sample region 16, the at least one target region 18, and the at least one channel 20 to realize a paper-based microfluidic device 10. Thus, such patterned barriers can define the shape and/or dimensions (e.g., width and length) of the channels 20, while the thickness of the paper defines the height of the channels 20. Aqueous solutions can be transported passively along the channels 20 by wicking through the hydrophilic fibers of paper. There are several methods to pattern the hydrophobic barriers on paper, such as photolithography, wax printing, PDMS application, and plasma treatment.

In one example, the hydrophobic material 14 is applied to a paper substrate 12 by wax printing. Patterns of the hydrophobic barriers can be designed using computer-aided design (CAD) software. The hydrophobic material barriers can then be printed (i.e., applied or patterned) onto the paper substrate 12 using a solid ink printer. The printed paper can then placed on a digital hot plate set at 150° C. for 120 s. When the wax on the surface of the paper melts, it spreads vertically as well as laterally into the paper. The vertical spreading can create the hydrophobic material barrier across the thickness of the paper.

The sample region 16 (FIG. 1) of the substrate 12 can be sized and dimensioned to receive a biological sample (e.g., saliva or whole blood). The sample region 16 can be defined by one or more side walls 102 that comprise the hydrophobic material 14 applied to the substrate to define the sample region 16 (FIG. 1). In one example, the sample region 16 can include a single side wall 102 that defines the sample region 16. The sample region 16 is in fluid communication with the channel 20. The sample region 16 (FIG. 2) can have a rectangular cross-sectional profile; although, other cross-sectional profiles are possible depending upon the number and shape of the side walls 102.

The sample region 16 can further comprise a predetermined amount of an agent suitable for treating the biological sample to facilitate operation of the immunoassay. For example in embodiments where a blood sample is used, the sample region 16 can include an agent capable of agglutinating red blood cells in a biological sample that permits blood plasma including at least one pathogen biomarker to pass into the channel 20 while preventing passage of larger cells (e.g., red blood cells). In one example, the agglutinating agent can include a predetermined amount of lectins that are applied to the sample region 16 of the substrate 12. Lectins for use as an agglutinating agent can include Concanavalin A, wheat germ agglutinin, and blue dextran.

In embodiments where the biological sample is a mucous-containing sample, such as a saliva sample, the sample region 16 can include a mucolytic agent in order to decrease the viscosity of the sample and encourage flow of the sample through the immunoassay. A variety of mucolytics are known to those skilled in the art. Examples of mucolytic agents include L-acetyl-cysteine, bromhexine hydrochloride, mecysteine, dornase alfa, and erdosteine.

The target region 18 is sized and dimensioned to provide the predetermined amount of antibodies 22 conjugated with colloidal nanoparticles. A preferred colloidal nanoparticle is colloidal gold. However, other types of colloidal nanoparticles such as silver nanoparticles or colored latex nanoparticles can be used in other embodiments. The target region 18 can be spaced apart from the sample region 16 and be in fluid communication with the channel 20. The target region 18 can be defined by one or more side walls 106 that comprise the hydrophobic material 14 applied to the substrate 12 to define an interior 46 of the target region 18. In one example, the target region 18 can include a single side wall 106 that defines a rectangular target region. The target region 18 (FIG. 2) can have a rectangular cross-sectional profile; although, other cross-sectional profiles are possible depending upon the number and shape of the side walls 106. The dimensions of the target region 18 can be the same as or different than the dimensions of the sample region 16.

The target region 18, or a portion thereof, can include a predetermined amount of antibodies 22 conjugated with colloidal gold nanoparticles. The predetermined amount of amount of antibodies 22 conjugated with colloidal gold nanoparticles can be coated onto and/or embedded in the target region 18 in a manner allowing for the biological sample to contact the antibodies in the target region 18 once the biological sample has flowed to the target region 18 through the channel 20 from the sample region 16. In some embodiments, the antibodies adsorb onto the colloidal gold nanoparticles without the need for a linking agent, while in other embodiments a linking agent is used.

In some instances, the target region 18 can include a control region 84. Examples of controls include positive controls and negative controls. A device 10 can include a positive control, a negative control, or both a positive and a negative control. A positive control indicates the immunoassay is functioning properly by providing a signal when an appropriate biological sample is applied, while a negative control indicates that immunoassay is functioning properly by providing no signal when an appropriate biological sample is applied. For example, a control region 84 serving as a positive control with can include colloidal particles (e.g., colloid gold particles) conjugated with antibodies that specifically bind to material normally in the biological sample to act as a positive control. For example, immunoglobulin G (IgG) is normally present in blood sample, so when blood samples are used, anti-IgG antibodies can be used in the positive control, while immunoglobulin A (IgA) is normally present in mucous-containing samples, so anti-IgA antibodies can be used in the positive control when mucous-containing samples are used. Alternatively, a control region 84 serving as a negative control can include colloidal particles (e.g., colloid gold particles) without any conjugated antibody.

The antibodies 22 conjugated with colloidal nanoparticles (e.g., colloidal gold nanoparticles) can comprise any antibodies capable of specifically binding to a targeted pathogen biomarker in a biological sample. Antibodies provided herein include polyclonal and monoclonal antibodies, as well as antibody fragments that contain the relevant antigen binding domain of the antibodies. Monoclonal antibodies may be produced in animals such as mice and rats by immunization. B cells can be isolated from the immunized animal, for example from the spleen. The isolated B cells can be fused, for example with a myeloma cell line, to produce hybridomas that can be maintained indefinitely in in vitro cultures. These hybridomas can be isolated by dilution (single cell cloning) and grown into colonies. Individual colonies can be screened for the production of antibodies of uniform affinity and specificity. Hybridoma cells may be grown in tissue culture and antibodies may be isolated from the culture medium. Hybridoma cells may also be injected into an animal, such as a mouse, to form tumors in vivo (such as peritoneal tumors) that produce antibodies that can be harvested as intraperitoneal fluid (ascites). The lytic complement activity of serum may be optionally inactivated, for example by heating.

Protocols for generating antibodies, including preparing immunogens, immunization of animals, and collection of antiserum may be found in Antibodies: A Laboratory Manual, E. Harlow and D. Lane, ed., Cold Spring Harbor Laboratory (Cold Spring Harbor, N.Y., 1988) pp. 55-120 and A. M. Campbell, Monoclonal Antibody Technology: Laboratory Techniques in Biochemistry and Molecular Biology, Elsevier Science Publishers, Amsterdam, The Netherlands (1984).

In some embodiments, the antibodies 22 can comprise biotin-conjugated antibodies, such as anti-human IgG-Biotin antibodies. The colloidal gold nanoparticles can also be conjugated to streptavidin, thereby allowing for the conjugation of the antibodies to the colloidal gold nanoparticles. Alternatively, the colloidal gold nanoparticles can also be conjugated to the antibodies through conventional conjugation chemistry reactions, such as but not limited to Click, EDC/NHS and adsorption reactions. See Jazayeri et al., Sensing and Bio-Sensing Research, 9, 17-22 (2016), the disclosure of which is incorporated herein by reference. Once a desired antibody is selected, a predetermined amount of the antibody 22 can be conjugated to colloidal gold in an attachment buffer prior to the antibodies 22 being provided in the target region 18. In one example, the antibodies 22 are conjugated to colloidal gold nanoparticles in an attachment buffer that comprises a 0.01×PBS solution. The amount of antibody conjugated to colloidal gold can vary depending on the conjugation method. Pathogen biomarker specific antibody can be conjugated to colloidal gold at a ratio of 5 ng-50 μg of anti-human IgG antibody per 100 μg colloidal gold. In one example, pathogen biomarker specific antibody can be conjugated to colloidal gold at a ratio of 5 μg of anti-human IgG antibody per 100 μg colloidal gold.

The predetermined amount of antibodies 22 can be the amount of the antibodies required to detect the presence of a targeted pathogen biomarker. The predetermined amount selected can be influenced by the sensitivity of the particular antibody to be utilized. In one example, a predetermined amount of the antibody 22 conjugated to colloidal gold can be provided in the target region 18 by slowly adding 2 μl of 50 μg/ml anti-Human IgG-colloidal gold nanoparticles to the targeting region 18 of the device 10, which is then allowed to dry for 20 minutes at room temperature.

The device 10 can include one or more channels 20 in fluid communication with the sample region 16 and/or the at least one target region 18. These channels provide a capillary force action to draw a portion of a biological sample placed on the sample region 16 to be drawn through the channel 20 and into contact with the testing region 18. The channels 20 can be defined by one or more side walls 108 that comprise the hydrophobic material 14 applied to the substrate to define an interior 48 of a channel 20 (FIG. 1). Each channel 20 can comprise any suitable path, passage, or duct through, over or along which materials (e.g., liquid, pathogen biomarkers, and/or reagents) may pass through the device 10. Each channel 20 may have any suitable dimensions and geometry, including width, height, length, and/or cross-sectional profile, among others, and may follow any suitable path, including linear, circular, and/or curvilinear, among others. In one example, the length of a channel 20 can be the length required to allow enough time during flow of the biological sample from the sample region 16 to the target region 18 for complete plasma separation from a whole blood biological sample. Each channel is of a suitable size for providing a desired flow rate of the biological sample. In one example, a width or diameter of each channel 20 can be less than about 100-90 microns, about 90-80 microns, about 80-70 microns, about 70-60 microns, about 60-50 microns, about 50-40 microns, about 40-30 microns, about 30-20 microns, about 20-10 microns, or about 10-5 microns, e.g., less than about 50 microns. The diameter or width of each channel 20 can be uniform across its length or may vary at one or more locations. The channel 20 may run parallel to the substrate 12 of the device, or in some embodiments, the channel 20 may be perpendicular to the substrate 12 and run through the substrate from one side of the device 10 to the other.

Each channel 20 also may have any suitable surface contour, including recesses, protrusions, and/or apertures, and may have any suitable surface chemistry or permeability at any appropriate position within the channel. Each channel 20 may branch, join, and/or dead-end to form any suitable network. Accordingly, a channel 20 may function in pathogen biomarker positioning, sorting, separation, retention, treatment, detection, propagation, storage, mixing and/or release, among others.

In some instances, the device 10 shown in FIGS. 1-2 can be configured as a multichannel device 70 (FIG. 3). A multichannel device 70 can therefore comprise the features or components of the device 10 shown in FIGS. 1-2 and described above, including one or more sample regions 76 and two or more targeting regions 78. In some instances, two or more targeting regions 78, 78′ can be arranged in an array around one sample region 76 in a pattern relative to the sample region 76. Thus, in one example, the targeting regions 78 are arranged in a circumferential pattern around the sample region 76.

A multichannel device 70 of the present disclosure also comprises two or more channels 80 in fluid communication with the one or more sample region 76 and the two or more target regions 78. The number of channels 80 can correspond to the number of targeting regions 78 of the multichannel device 70. Thus, in one example, a multi-channel device 70 (e.g., a two-channel device) can include a second channel 80′ that is arranged radially opposite the other channel 80. The two-channel device can also comprise a second targeting region 78′ for providing the predetermined amount of antibodies 82 conjugated to colloidal gold. The second channel 80′ can be in fluid communication with the sample region 76 and the second targeting region 78′.

Having multiple channels advantageously provides the ability to probe multiple biomarkers in a single biological sample. In one example, a multichannel device 70 can include four channels 80; however, it will be appreciated that the device can include any number of channels 80 (e.g., two, three, five, or more). Each channel 80 of the multichannel device 70 can be radially spaced apart in relation to the sample region 76. Each channel 80 of the multichannel device 70 can be either equally or variably spaced apart from the other channels 80.

In another aspect, all or only a portion of the channel 20 can be coated with one or more capture antibodies that can specifically bind to a pathogen biomarker as the biological sample moves through the channel 20. As the biological sample enters into the target region 18, the predetermined amount of antibodies 22 conjugated with colloidal nanoparticles (e.g., colloid gold nanoparticles) can then specifically bind to the capture antibody bound to the pathogen biomarker, thereby allowing visual detection of the presence of the pathogen biomarker in the biological sample. In one example, the capture antibodies are anti-human IgG antibodies that specifically bind to IgG antibodies produced by a human in response to the presence of a pathogen.

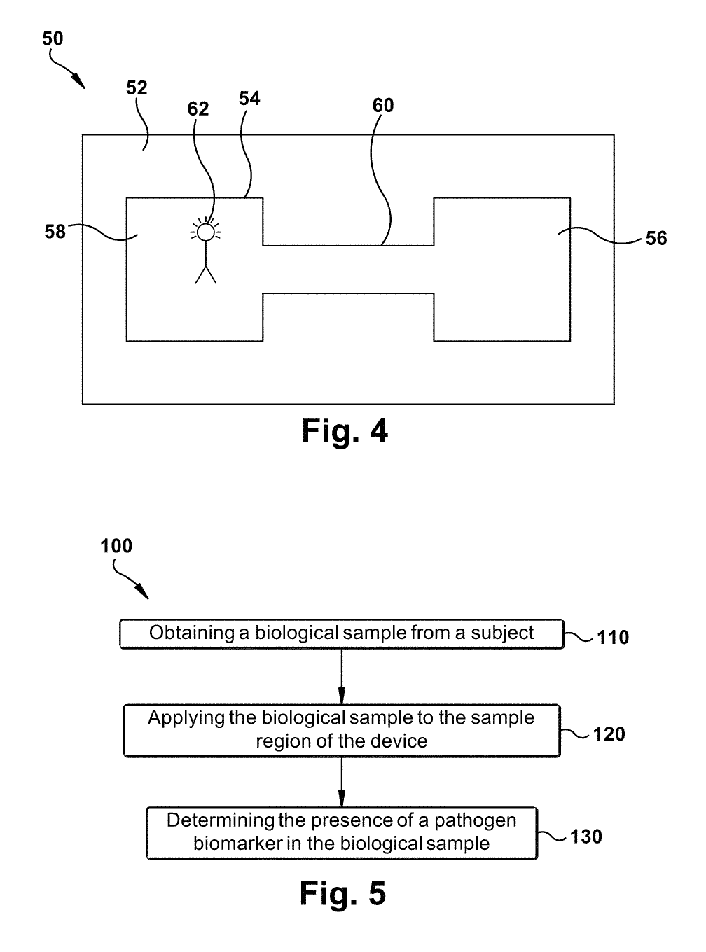

FIG. 4 illustrates a device 50 for detecting a target pathogen biomarker in a biological sample constructed in accordance with another aspect of the present disclosure. Except where described below, the device 50 can be identically constructed as the device 10 shown in FIGS. 1-2. For example, the device 50 can comprise a substrate 52, a hydrophobic material 54 applied to the substrate 52, and a predetermined amount of antibodies 62 that specifically bind to at least one pathogen biomarker in the biological sample. The predetermined amount of antibodies 62 is conjugated with colloidal gold nanoparticles. As described for the device 10, the hydrophobic material 54 applied to the substrate 52 defines a sample region 56 for receiving a biological sample, at least one target region 58 for providing the predetermined amount of antibodies 62, and at least one channel 60 that is in fluid communication with both the sample region 56 and the target region 58.

In use, a biological sample can be loaded into the sample region 56 of the device 50. The biological sample can then move from the sample region through the channel 60 and into the target region 58 via capillary action. As the biological sample moves into the target region 58, the biological sample is contacted with the predetermined amount of antibodies 62 conjugated with colloidal gold nanoparticles provided by the target region 58. If the at least one pathogen biomarker is present in the biological sample, the pathogen biomarker specifically binds to the predetermined amount of antibodies 62 conjugated with colloidal gold in the target region 58. The specific binding of the antibodies conjugated with colloidal gold 62 to the pathogen biomarkers results in the formation of colloidal gold conglomerates.

Colloidal nanoparticles create a signal as a result of clumping of the nanoparticles as a result of binding of the conjugated antibodies to their target antigen. The interaction of the colloidal gold nanoparticles with light is strongly influenced by their physical size, dimensions and environment. As free nanoparticles (NPs) colloidal gold nanoparticles will absorb light largely in blue-green (about 450 nm) color spectrum of visible light and will strongly reflect red light (about 700 nm), thus accounting for the deep maroon red color of free colloidal gold nanoparticles suspensions. However, as colloidal gold nanoparticles are brought into close proximity with each other and particle size increases (e.g., the conglomeration of colloidal gold nanoparticles due to the binding of these to an targeted pathogen biomarker via a conjugated antibody), the absorption spectrum of the colloidal gold nanoparticles shifts to red light causing blue light to be reflected instead, thus inducing a visible shift in color from red/maroon to blue/purple.

Therefore, a conglomeration of colloidal gold is detected by the observation of a change in the visible color spectrum from red to purple in a portion of the targeting region providing the antibodies conjugated with colloidal gold 62. For example, if at least one pathogen biomarker is present in the biological sample, the observation of a change in the visible color spectrum can occur in about 20-25 minutes (e.g., about 1-5 minutes, about 5-10 minutes, about 10-15 minutes, about 15-20 minutes, or about 20-25 minutes, such as 22 minutes or about 22 minutes). Alternatively, if the at least one pathogen biomarker is not present in the biological sample, the pathogen biomarker will not bind to the predetermined amount of antibodies 62 conjugated with colloidal gold and the visible color spectrum of the targeting region 58 will remain red.

It will be appreciated that the order of steps involved in operation of the device 52 may be changed, or that certain steps may be omitted depending upon the particular application. For example, the biological sample could be filtered to remove certain particles (e.g., red blood cells and cell debris) or treated to reduce viscosity prior to loading into the sample region 56. Alternatively, a lysing solution could be added to the biological sample before loading into the sample region 56. In addition, a predetermined amount of an agent such as agglutinating agent or a mucolytic agent could be provided in the sample region 56 or added to the biological sample prior to loading the biological sample into the sample region.

### Methods

Another aspect of the present disclosure can include a method 100 (FIG. 5) for detecting a pathogen in a biological sample. In one example, the method 100 can be performed using the device 10 illustrated in FIGS. 1-2 and described above. Generally, the method 100 can include the steps of: obtaining a biological sample from the subject (step 110); applying the biological sample to the sample region 16 of the device 10 (step 120); and determining the presence of the pathogen biomarker in the biological sample (step 130). The method 100 can find use in a variety of settings and with a number of applications, such as use in a point-of-care environment or for high-throughput analysis. For example, operation of the device 10 can be accomplished or assisted using a conventional automated colorimetric analysis machine.

At Step 110 of the method 100, the biological sample can be obtained from a subject using suitable conventional means. For example, a whole blood biological sample can be withdrawn from a subject using a hypodermic needle. At Step 120 of the method 100, a biological sample (e.g., a liquid sample) can be applied to the sample region 16 of the device 10. The biological sample can be previously withdrawn from a subject using a hypodermic needle at Step 110, for example, and then applied directly into the sample region 16 by dispensing the liquid sample into the interior 38 of the sample region 16 at Step 110. Alternatively, the biological sample can be pre-processed (e.g., centrifuged, contacted with one or more reagents, etc.) prior to applying the biological sample into the sample region 16.

Once the biological sample is applied to the sample region 16 of the device 10, the biological sample can then move/flow from the sample region 16 through the channel 20 into the target region 18 via capillary action. As the biological sample moves/flows into the target region 18, the biological sample is contacted with the predetermined amount of antibodies 22 conjugated with colloidal gold nanoparticles located in the target region 18.

At Step 130 of the method 100, the presence of at least one pathogen biomarker in the biological sample can be determined. If the at least one pathogen biomarker is present in the biological sample, the pathogen biomarker specifically binds to the predetermined amount of antibodies 22 conjugated with colloidal gold in the target region 18. The specific binding of the antibodies conjugated with colloidal gold 22 to the pathogen biomarkers results in the formation of colloidal gold conglomerates. The conglomeration of colloidal gold is detected by the observation of a change in the visible color spectrum from red to purple in a portion of the targeting region providing the antibodies conjugated with colloidal gold 22. Alternatively, if the at least one pathogen biomarker is not present in the biological sample, the pathogen biomarker will not bind to the predetermined amount of antibodies 22 conjugated with colloidal gold and the visible color spectrum of the targeting region 18 will remain red.

In some aspects, pathogens that may be detected using method 100 described herein include, but are not limited to, viruses endemic to resource-limited regions, such as all four serotypes of dengue virus, West Nile virus, yellow fever, and Ebola virus. The presently described device can also be used to detect pathogens related to common sexually transmitted disease. Devices and methods described herein may be further applicable to, and useful for, determining the presence of pathogen biomarkers in water sources, vegetation, food production, and many other applications where protein-based assays may be employed. For example, in some embodiments, the devices and methods can be used to detect flaviviruses such as bovine viral diarrhea virus (BVDV) that can be found in livestock.

In some instances, a pathogen biomarker detected using a device and method of the present disclosure is a pathogen biomarker related to and/or indicative of the presence of dengue virus in a subject. The dengue virus detected using a device in accordance with a method described herein can include one of four antigenically and genetically distinct virus serotypes, designated dengue-1, -2, -3 and -4, and combinations thereof. The existence of four different viruses that cause dengue illness has previously been a major roadblock in the development of laboratory and rapid diagnostics to detect dengue infection. The biomarkers of dengue virus infection also differ during the febrile, critical, and recovery phases of the disease progression. Thus, the presence of a certain dengue pathogen biomarkers as determined in a method of the present disclosure can be indicative of the disease progression of a dengue infection in a subject. For example, detecting the presence of human anti-dengue IgG and/or IgM antibodies in a biological sample, which are only present in the subject 7-15 days after primary infection, can indicate a later critical and/or recovery phase of dengue infection in the subject. On the other hand, detecting the presence of dengue NS1 antigen in a biological sample, which is only present in the early stages of infection, can indicate a febrile phase of dengue infection in the subject. Therefore, methods of the present disclosure can lead to an early detection of dengue infection in a subject, thereby advantageously providing a better chance of managing the disease.

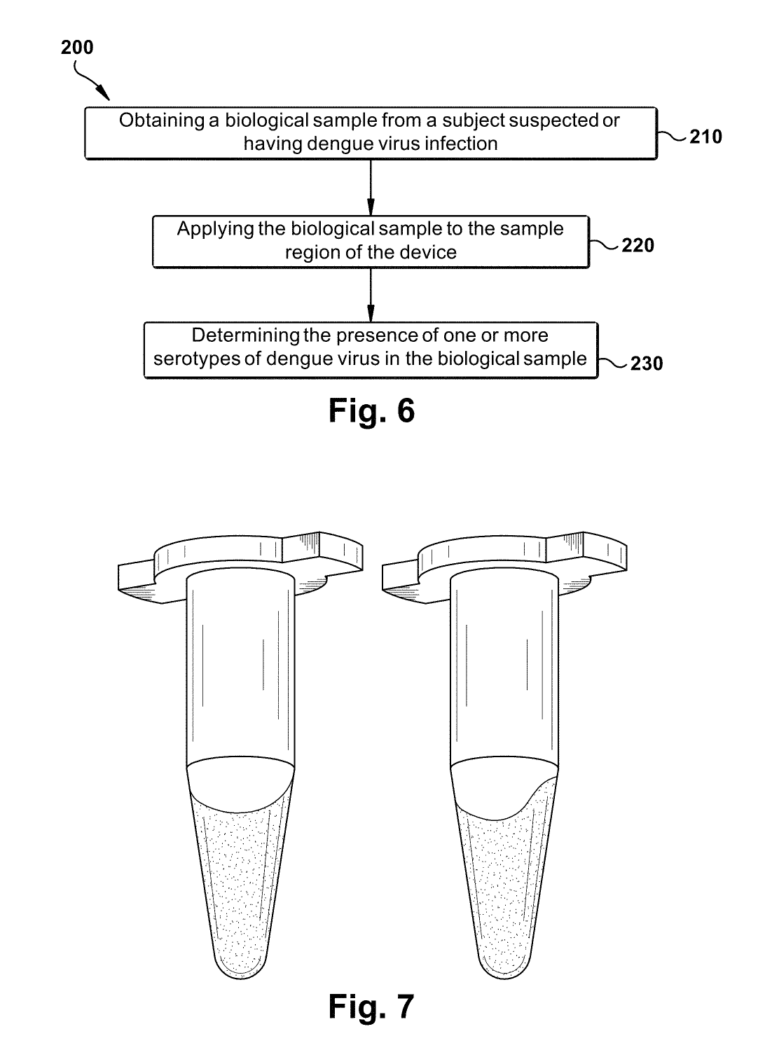

Thus, in one example, the present disclosure can comprise a method 200 (FIG. 6) for detecting the presence of a dengue virus in subject using a multichannel device 70 described above (FIG. 3). Generally, the method 200 can include the steps of: obtaining a biological sample from the subject (step 210), wherein the subject is suspected of having a dengue virus infection; applying the biological sample to the sample region 76 of the device 70 (step 220); and determining the presence of one or more serotypes of dengue virus in the biological sample (step 230).

At Step 210 of the method 200, the biological sample can be obtained from a subject using suitable conventional means. For example, a whole blood biological sample can be withdrawn from a subject using a hypodermic needle. At Step 220 of the method 100, a biological sample (e.g., a whole blood liquid sample) can be applied to the sample region 76 of the device 70. The biological sample can be previously withdrawn from a subject using a hypodermic needle at Step 210, for example, and then applied directly into the sample region 76 by dispensing the liquid sample into the sample region 76 at Step 210. Alternatively, the biological sample can be pre-processed (e.g., centrifuged, contacted with one or more reagents, etc.) prior to applying the biological sample into the sample region 76.

Once, the biological sample is applied to the sample region 76 of the device 70, the biological sample can then move/flow from the sample region 76 through the channels 80 and into the target regions 78 via capillary action. As the biological sample moves/flows into the target regions 78, the biological sample is contacted with the predetermined amount of antibodies 82 conjugated with colloidal gold nanoparticles provided by the target regions 78. Each target region of the multichannel device 70 can provide antibodies to biomarkers for different dengue virus serotypes (e.g., dengue-1, -2, -3, and -4). For example, a multichannel device 70 can include four or more target regions, wherein a first target region 78 can include a predetermined amount of antibodies that can specifically bind to a dengue-1 serotype biomarker, a second target region 78′ can include a predetermined amount of antibodies that can specifically bind to a dengue-2 serotype biomarker, a third target region 78″ can include a predetermined amount of antibodies that can specifically bind to a dengue-3 serotype biomarker, and a fourth target region 78′″ can include a predetermined amount of antibodies that can specifically bind to a dengue-4 serotype biomarker. The multichannel device 70 can further include a control target region 84 providing a positive and/or negative control. For example, the positive control can colloidal gold particles conjugated with anti-human IgG as described above to indicate proper function of the device 70.

At Step 230 of the method 200, the presence of at least one pathogen biomarker in the biological sample can be determined. If the at least one pathogen biomarker is present in the biological sample, the dengue serotype pathogen biomarker specifically binds to the predetermined amount of serotype specific antibodies 82 conjugated with colloidal gold in the one or more of the target regions 78. The specific binding of the antibodies conjugated with colloidal gold 82 to the dengue serotype pathogen biomarkers results in the formation of colloidal gold conglomerates. The conglomeration of colloidal gold is detected by the observation of a change in the visible color spectrum from red to purple in a portion of the targeting region 78 providing the antibodies conjugated with colloidal gold 82. Alternatively, if a particular dengue serotype pathogen biomarker is not present in the biological sample, the pathogen biomarker will not bind to the predetermined amount of serotype specific antibodies 82 conjugated with colloidal gold and the visible color spectrum of the targeting region 78 will remain red.

The following examples are for the purpose of illustration only and are not intended to limit the scope of the claims, which are appended hereto.

### EXAMPLES

### Example 1: Determining the Presence of Pathogen in a Biological Sample Using a Microfluidic Device

Here, a point-of-care paper-based microfluidic rapid diagnostic device is used to screen for multiple viruses with a single, low-volume blood sample. Microchannels are patterned on nitrocellulose paper using a solid ink printer, which allows the passive flow of liquid to be directed to specific areas of the device. Due to the small size of the biological sample used during the assay, a sensitive colorimetric indicator derived from colloidal gold is used to interpret the results.

A small blood sample is applied to the middle of the device, then plasma is passively separated from the red blood cells through a process known as hemagglutination. The plasma containing the viruses passively flows to each specific area or detection zone that is pre-treated with colloidal gold conjugated with pathogen-specific antibodies. If the sample contains any pathogen proteins detected by the antibodies, a chemical reaction is triggered to induce a colorimetric change.

### Conjugation of Antibodies Abs to AuNPs in 0.01×PBS Attachment Buffer

Protocol for conjugation of biotinylated antibodies to streptavidin-AuNPs:

1. 100 μg of 40 nm Streptavidin-AuNPs (NanoComposix) were added to 10 μg anti-Human IgG-Biotin (BioLegend) in a total volume of 500 μl 0.01× Phosphate-Buffered Saline (PBS).
2. Sample was incubated at room temperature with rotation for 1 hour in the dark.
3. AuNPs were recovered following conjugation by centrifugation at 3,600 g×10 minutes at room temperature.
4. AuNPs were washed two times in 0.01×PBS before being re-suspended to a working concentration of 100 μg/ml.
5. Conjugated AuNPs were stored at 4° C. in the dark until further use.

As shown in FIG. 7, un-reacted AuNPs (left) appear the same as anti-Human IgG conjugated AuNPs (right) following conjugation in 0.01×PBS. Therefore, 0.01×PBS is an appropriate medium for conjugation of antibodies to AuNPs while maintaining the appearance of the particles in suspension.

Particle Structure after Conjugation

Although the physical appearance of AuNPs was identical to un-reacted AuNPs after conjugation the nano-structure of both required comparison to ensure the conjugation process did not adversely interfere with the AuNPs' physical composition.

Protocol for transmission electron microscope (TEM) imaging:

1. Conjugated and un-conjugated particles were mounted onto copper TEM grids (Fischer) and allowed to dry.
2. Samples were negatively stained twice in 20 μl 1% filtered-urynal acetate solution (Sigma Aldrich).
3. Slides were imaged on a Tecnai G2 F30 TWIN TEM microscope.

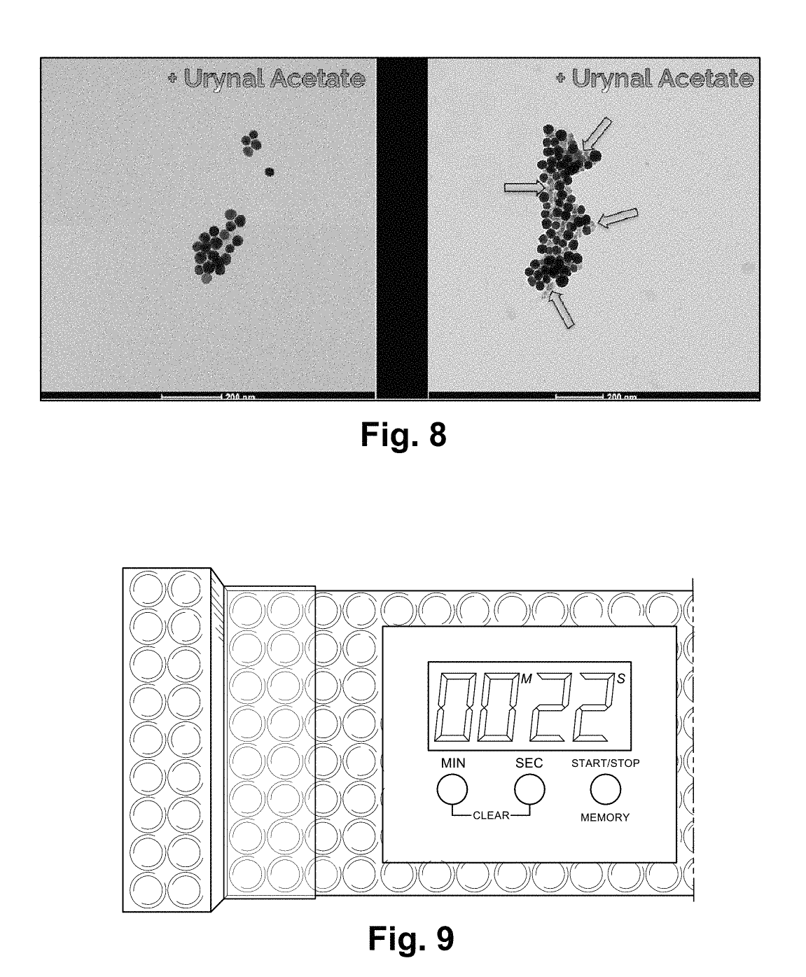

Therefore, as shown in FIG. 8, the structure of AuNPs is not altered following conjugation to anti-Human IgG (right) compared to un-conjugated AuNPs (left). Darkened areas around the anti-human IgG-conjugated AuNPs suggests successful attachment of the Ab to the NPs. the use of 0.01×PBS is a suitable medium for the attachment of biotin-conjugated antibodies to streptavidin-AuNPS.

### In Vitro Detection of Human IgG Using AuNPs.

In order to determine that anti-Human IgG-AuNPs can recognize and initiate a color change upon the binding to human IgG an in vitro test of the particles was performed.

Protocol for in vitro testing:

1. High-binding 96 well flat bottomed microtiter plates (NUNC) were coated with 50 μl of 1 mg/ml and subsequent decreasing (1:2 dilutions) concentrations of purified human IgG (Sigma Aldrich) in 1×PBS for 2 hours at 37° C.
2. Plates were washed three times with 1×PBS-Tween and tapped dry.
3. 100 μl of 5% Bovine Serum Albumin (BSA) (Sigma Aldrich) was added to each well for 1 hour at 37° C. to block plates against non-specific interactions leading to false positives.
4. Plates were washed three times with 1×PBS-Tween and once with 0.01×PBS and tapped dry.
5. 50 μl of 50 μg/ml un-conjugated AuNPs or anti-Human IgG-conjugated AuNPs were added to each well and plates observed for a visible color change.

As shown in FIG. 9, anti-human IgG conjugated AuNPs reacted (color change to purple) after 22 seconds to all concentrations of Human IgG while un-conjugated particles did not (remained red). This indicates the specific recognition and binding to of anti-Human IgG-AuNPs to Human IgG while un-conjugated AuNPs did not. Moreover, the levels of Human IgG assayed in this test fall well below those normally found in human blood, demonstrating the high level of sensitivity of the assay.

### Example 2: Additional Immunoassays

Bovine viral diarrhea virus (BVDV) is a RNA virus in the flavivirus family that causes a highly complex immune-suppressing disease, which is considered to be the most costly viral disease in U.S. cattle herds. BVDV tends to reduce the reproduction rate in livestock and milk production in dairy cows. The disease is mainly transmitted by persistently infected (PI) animals, which carry the virus and shed large amounts of it through their lifetime. PI animals tend to increase the chance of cattle contracting BVD by up to 43% when present in the same feedlots as other cattle. The economic burden of BVDV on feedlot cattle, which can house up to 10,000s of animals, is estimated at around $67.49 per animal. BVDV also gives rise to bovine respiratory disease (BRD) due to its immune suppression leading to an estimated 50 to 75% mortality. BVDV causes an annual loss of $20 million per million calvings to the cattle industry leading to a $93 million loss in 2015.

Current methods of testing cattle usually involve pooled blood or ear-notch samples from the herd to test for BVDV using enzyme-linked immunosorbent assays (ELISA). These tests, as well as current rapid diagnostic tests, cannot discern between PI animals and transiently infected (TI) animals, and usually take a week leaving the possibility of more animals in the herd being infected. Additionally, this testing typically takes place within a laboratory environment that requires collecting and shipping of samples from the source of origin. Our test is intended at the point-of-care either in feedlot operations or livestock markets where animals can be quickly screened and evaluated before being circulated throughout the herd where the BVD virus can quickly spread.

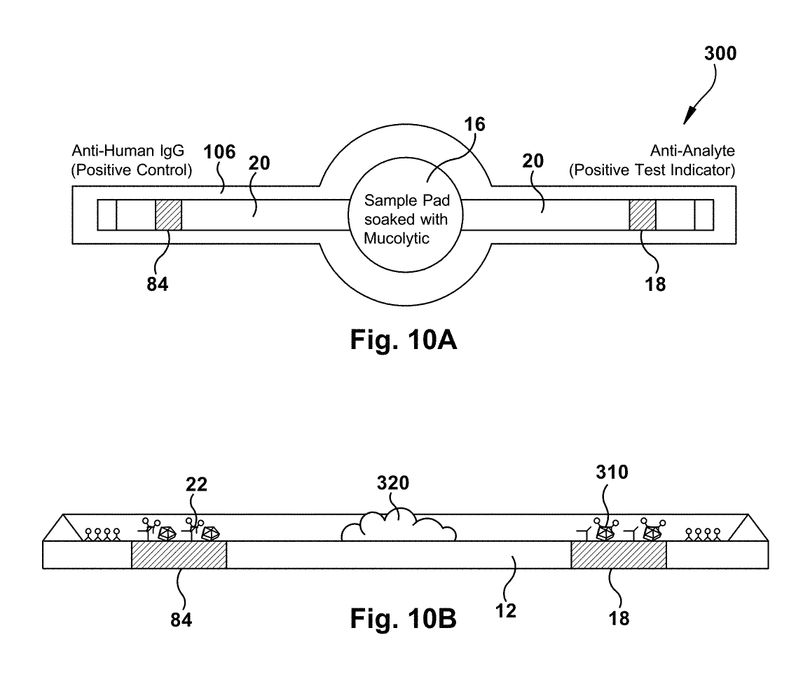

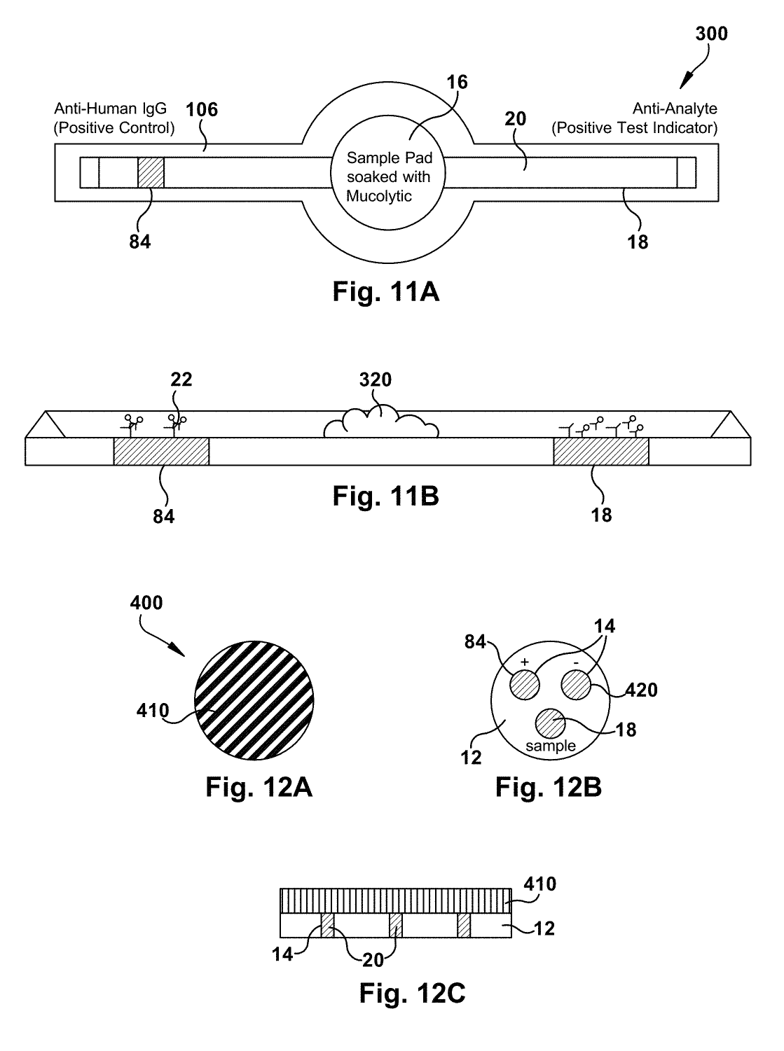

The inventors have developed a number of different immunoassays including control regions and sample regions. FIG. 10 provides a top (10A) and side (10B) view of an immunoassay including a sample pad and a positive control, showing a positive result indicating that virus is present. FIG. 11 provides a top (10A) and side (10B) view of the same assay, showing a negative result indicating that the target virus is absent.

FIGS. 10 and 11 show an embodiment of a multichannel device 300 in which a sample region 16 is provided in the middle of the device with two channels 20 leading to an anti-analyte test region 18 and a control region 84, all positioned in parallel over a substrate 12. The channel 20 on the left of the figures leads to the positive control 84 which has gold nanoparticles conjugated with antibodies 22 to the target antigen (e.g., anti bovine immunoglobulin A (IgA)). The channel on the right of the figures leads to a target region 18 which has gold nanoparticles conjugated with antibodies 22 against the target antigen (e.g., anti-BVDV antibodies). The antibodies bind to the target virus 310, causing the colloidal gold nanoparticles to generate a signal. In this embodiment, the sample region 16 includes a sample pad 320 containing a mucolytic. The mucolytic will loosen the mucous of mucous-containing samples such as those typically used to detect BVDV before it goes into the left and right channels. As shown in FIG. 10, the positive control will always change color from red to purple as mucus always has IgA and this will indicate to the user about what is a positive test. The target region 18 in channel 20 on the right will only change color if the mucous sample has the BVDV virus, with FIG. 10 showing a positive result and FIG. 11 showing a negative result. The diagram just shows that the gold nanoparticles bound to the antibodies will conglomerate and bind to the antigen if it is present. That causes the color change from red to purple.

FIG. 12 shows another embodiment of a multichannel device 400, including a top view (12A), a bottom view (12B), and a side view (12C). This embodiment of the immunoassay includes both positive and negative controls, and channels that run perpendicular to the substrate of the device. The device 400 includes a substrate (e.g., a paper substrate) 12 attached to an absorbent pad 410 with hydrophobic material (e.g., wax) 14 forming channels 20 running through both the substrate 12. This embodiment is particularly suitable for use with mucous-containing biological samples. The biological sample is applied to the absorbent pad and directly transfers to channels 20 which form target and control regions (e.g., the three rings shown), or more specifically a target region 18, a positive control 84, and a negative control 420.

Mucous samples from the animal will be applied to the absorbent pad (FIG. 12A) which can include a mucolytic agent (e.g., L-acetyl-cysteine) already saturated within the absorbent material and will physically breakdown the mucous so it will easily flow through the absorbent pad. Once, the mucous has reached the paper substrate it will fill three different channels 20 (shown in side view FIG. 12C) created by the embedded hydrophobic material 14. Each region (18, 84, and 420) consists of colloidal nanoparticles with specific biomarkers bound to them so that when a complimentary biomarker from the mucous sample is detected within the region a chemical reaction will occur resulting in a change of color. The colorimetric result can be evaluated by viewing target and control regions on the bottom of the device (FIG. 12B) by indicating if the test is still reliable from the positive and negative controls, as well as indication of viral presence in the sample region.

The complete disclosure of all patents, patent applications, and publications, and electronically available material cited herein are incorporated by reference. The foregoing detailed description and examples have been given for clarity of understanding only. No unnecessary limitations are to be understood therefrom. The present disclosure is not limited to the exact details shown and described, for variations obvious to one skilled in the art will be included within the present disclosure defined by the claims.

What is claimed is:

1. A microfluidic device comprising:
(a) a substrate;
(b) a hydrophobic material applied to the substrate to define at least one target region, a sample region for receiving a biological sample, and at least one channel in fluid communication with the at least one target region and the sample region; and
(c) a predetermined amount of antibodies that specifically bind to at least one pathogen biomarker provided in the at least one target region, wherein the predetermined amount of antibodies is conjugated with colloidal gold.

2. The device of claim 1, wherein the at least one pathogen biomarker comprises a pathogen antigen.

3. The device of claim 1, wherein the at least one pathogen biomarker comprises a mammalian antibody that specifically binds to a pathogen antigen.

4. The device of claim 1, wherein the antibodies comprise biotin conjugated antibodies and the colloidal gold comprises streptavidin conjugated colloidal gold.

5. The device of claim 4, wherein binding of the antibodies conjugated with colloidal gold to the at least one pathogen biomarker is indicated by a change in the visible color spectrum from red to purple.

6. The device of claim 1, wherein the substrate comprises paper.

7. The device of claim 1, wherein the hydrophobic material comprises wax.

8. The device of claim 1, further comprising a positive control.

9. The device of claim 8, wherein the positive control comprises colloidal gold particles conjugated with anti-human IgG or anti-human IgA.

10. The device of claim 1, further comprising a negative control.

11. The device of claim 1, further comprising a positive control and a negative control.

12. The device of claim 1, wherein the at least one pathogen biomarker comprises at least one viral pathogen biomarker.

13. The device of claim 12, wherein the at least one viral pathogen is selected from the group consisting of dengue-1 virus, dengue-2 virus, dengue-3 virus, dengue-4 virus, and combinations thereof.

14. The device of claim 1, wherein the viral pathogen is bovine viral diarrhea virus.

15. The device of claim 1, wherein the at least one pathogen related biomarker comprises at least one bacterial pathogen biomarker.

16. The device of claim 1, being configured as a multichannel device and further comprising:
two or more target regions, and two or more channels in fluid communication with the two or more target regions to the sample region.

17. A method of detecting the presence of at least one pathogen in a subject comprising:
(a) obtaining a biological sample from the subject;
(b) applying the biological sample to a microfluidic device comprising
(i) a substrate; wherein the substrate comprises a sample area and a targeting area;
(ii) a hydrophobic material applied to the substrate to define at least one target region, a sample region for receiving a biological sample, and at least one channel in fluid communication with the at least one target region and the sample region; and
(iii) a predetermined amount of antibodies that specifically bind to at least one pathogen biomarker provided in the at least one target region, wherein the predetermined amount of antibodies is conjugated with colloidal gold; and
(c) determining the presence of the at least one pathogen biomarker in the biological sample, wherein the at least one pathogen biomarker, if present, specifically binds to the predetermined amount of antibodies conjugated with colloidal gold in the target region causing the formation of a colloidal gold conglomerate, and wherein the presence of a colloidal gold conglomerate is indicative of the presence of the at least one pathogen in the subject.

18. The method of claim 17, wherein the formation of the colloidal gold conglomerate is indicated by a change in the visible color spectrum from red to purple.

19. The method of claim 17, wherein the antibodies comprise biotin conjugated antibodies and the colloidal gold comprises streptavidin conjugated colloidal gold.

20. The method of claim 17, wherein the substrate comprises paper.

21. The method of claim 17, wherein the hydrophobic material comprises wax.

22. The method of claim 17, wherein the at least one pathogen biomarker comprises at least one viral pathogen biomarker.

23. The method of claim 22, wherein the at least one viral pathogen biomarker is selected from the group consisting of dengue-1 virus, dengue-2 virus, dengue-3 virus, dengue-4 virus, and combinations thereof.

24. The method of claim 22, wherein the viral pathogen comprises bovine viral diarrhea virus.

25. The method of claim 17, wherein the at least one pathogen biomarker comprises at least one bacterial pathogen biomarker.

26. The method of claim 17, wherein the biological sample is selected from the group comprising blood, urine, saliva, semen, perspiration, and mucus.

27. The method of claim 17, wherein the subject is domesticated livestock.

28. The method of claim 17, wherein the subject is a human.

29. The method of claim 17, wherein the device is configured as a multichannel device and further comprises two or more target regions, and two or more channels in fluid communication with the two or more target regions to the sample region.
